(19) United States
(12) Patent Application Publication
(10) Pub. No.: US 2010/0070037 A1
(43) Pub. Date: Mar. 18, 2010

(54) ORTHOPAEDIC IMPLANTS AND PROSTHESES

(76) Inventors: John Parry, Worcestershire (GB); Dan Purdue, Worcestershire (GB); Graham Aitchison, Worcestershire (GB); Larry T. Khoo, Worcestershire (GB)

Correspondence Address: NIXON & VANDERHYE, PC, 901 NORTH GLEBE ROAD, 11TH FLOOR, ARLINGTON, VA, 22203, US

(21) Appl. No.: 12/312,714

(22) PCT Filed: Oct. 31, 2007
(86) PCT No.: PCT/GB2007/050668

§ 371 Date: May 22, 2009

(30) Foreign Application Priority Data
Nov. 29, 2006 (GB) 0623801.8

(51) Int. Cl. A61F 2/44

(52) U.S. Cl. 623/17.16

(57) ABSTRACT

The present invention provides a spinal implant or prosthesis having a plate portion for securing to adjacent vertebrae and a cage portion for insertion therebetween in which one or more plate portions are pivotally connected to the cage portion. Preferably, both the upper and lower portions are pivotally connected to rotate about a common axis (X).

The present invention relates to orthopaedic implants and prostheses and relates particularly but not exclusively to implants and prostheses for bone structures, particularly in the cervical, thoracic and lumbar spine regions.

### BACKGROUND ART

Bones and related structural body parts, for example spine and/or vertebral bodies and/or inter-vertebral discs, may become crushed or damaged as a result of trauma/injury, or damaged by disease (e.g. by tumour, auto-immune disease), or damaged as a result of degeneration through an aging process. In many such cases the structure can be repaired by replacing the damaged parts (e.g. vertebra and/or discs) with a prosthesis or implant. A method of repair is to remove the damaged part(s) (e.g. vertebra and/or partial vertebra and/or disc and/or partial disc) and replace it with an implant or prosthesis such that the implant or prosthesis is free standing or fastened in position between adjacent undamaged parts (e.g. adjacent vertebral bodies).

Associated with this method of repair, is fusion of the bone structure where the implant or prosthesis is placed. Typically an implant or prosthesis may consist of a central space surrounded by a continuous wall that is open at each end (e.g. superior and inferior). This form of implant or prosthesis is thought to allow bone to develop within the central space, developing from each extremity of the implant or prosthesis towards the centre. Typically an implant or prosthesis is secured directly to a bone structure by mechanical or biological means.

Many current implants and prostheses are hollow to allow bone growth within the hollow space. One problem, when replacing large structural sections, is that the relationship of length (or height) to cross sectional area of the central space is large. The larger this relationship, the more problems arise in providing an adequate blood and nutrient supply to allow fusion and or bone growth into the hollow centre, either in a timely manner, or at all. One solution to this problem is to make the central space with as large a cross section as possible. However, this is limited by the wall thickness and the material used for the implant or prosthesis, which determines its mechanical strength. For this reason, orthopaedic surgeons often pack the space within the implant or prosthesis with an injectable or mouldable bone growth encouraging material or with fragments of bone taken from other parts of the patients body i.e. autograft or bone from biocompatible sources, for example allograft or synthetic bone. Even then there may not be complete fusion of the implant or prosthesis into the bone structure.

One problem with metal implants or prostheses is that the Modulus of Elasticity is much higher than the bone structure to which it is secured. This creates a relatively higher stiffness resulting in stresses being transferred to adjacent bone structures, for example an adjacent vertebra and potential stress fractures through stress shielding and bone graft resorbtion.

Another problem is that the implant or prosthesis is generally not sufficiently secured to the spine and does not provide sufficient stability of the spine when used alone. To achieve this necessary stability, the implant or prosthesis requires a second system such as a plate or rod based system that is attached to the spine and the implant or prosthesis. This additional system is not integrated, it is an additional cost, and it may increase the operative time and risk to the patient.

When a cage is combined with a plate covering the cage that is used to lock adjacent vertebrae and for the plate to match the patient's anatomic shape it can be difficult to install the combined assembly and ensure optimal vertebrae movement. Indeed, installation of the cage itself can be problematic particularly when access is difficult or the profile of the vertebrae prevents easy insertion. Another problem is that cages with plates are generally rigid in structure and are designed not allow relative movement between plate and cage or adjoining vertebral bodies and cage specifically in flexion and extension of the spine.

Another problem is that Surgeons have different viewpoints in the way that this method of repair is carried out. Viewpoints range from rigid fixation to semi-rigid fixation. Currently the Surgeon would need to select different products and different sets of instrumentation to provide this choice whereas the invention allows the choice with one implant and one set of instrumentation.

The implant or prosthesis is attached to the adjacent vertebral body using a fixing e.g. a screw. A problem generally with such fixing or fixing systems is that, after insertion into the vertebral body, the fixing can work itself loose and/or back-out i.e. withdraw from the vertebral body. The consequence of back-out or loosening of the implant or prosthesis includes loss of stability, potential risk to the patient and a separate costly and often painful operation.

Another problem is that implants or prostheses are generally manufactured from materials that are structurally acceptable but remain in the body for an indefinite period. Such metal implants or prosthesis designed for fusion have a Young's modulus greater than natural bone that may result in mechanical stress shielding in adjacent levels, leading to high stresses, deformation and/or fractures of the adjacent vertebral body.

The present invention provides a spinal implant comprising upper and lower plate portions for securing to vertebral bodies, wherein one or more of said plate portions are pivotal about an axis such as to allow for flexibility between vertebrae.

Preferably, the arrangement includes a resistance means for resisting pivotal movement of said one or more upper or lower portions and wherein one or more of said plate portions includes an engagement surface with which said resistance means engages.

Advantageously, said implant includes a mounting portion and wherein said resistance means comprises a bolt having an engagement portion for frictional engagement with said one or more plate portions and a bolt thread for engagement with said mounting portion. Said resistance means may comprise a bolt head portion of said bolt and said one or more plates include a channel having edges therein which form an engagement surface and which, in operation, engage with said bolt head. One or more of said plate portions may include a semi-circular collar portion, said axis comprises a rod portion and said semi-circular collar portion engages around said rod portion for pivotal movement relative thereto. Preferably, said upper and lower plate portions each include semi-circular collar portions and each engages with said rod portion for pivotal movement relative thereto. The arrangement preferably includes a mounting boss for receiving the bolt thread of said bolt and said boss includes a rod portion onto which one or more of said plate portions are mounted for pivotal movement relative thereto.

Advantageously, the one or more engagement surfaces on the one or more plate portions are on an inner surface of said plate or plates.

In a particularly advantageous arrangement one or more of said upper or lower plates include a “click-fit” fitting for engagement with said rod.

Preferably, said upper and lower plates include a cut-out portion on confronting edges thereof, said bolt head includes an axis which extends through said cut-out portion and includes an engagement feature accessible through said cut-out portion and said bolt head has a diameter greater than the size of said cut-out. Conveniently, said upper plate and said lower plate each comprise a pair of collar portions and wherein one pair are spaced apart more than the other pair such as to allow one pair to nestle between the other pair when engaged with said rod.

In an assembled state the implant includes a cage portion for insertion between vertebrae.

In a particularly advantageous arrangement said upper or lower plate portions pivot about a common axis X.

The cage may comprise a replacement cage for replacing a vertebra and said cage may include upper and lower rod portions.

Advantageously, said cage portion comprises an open ended cage having side arms and wherein said side arms each include a coupling for coupling said cage to said plates.

Preferably, the implant includes a rod portion about which said plates pivot and said coupling comprises one or more cut-outs for engagement with the rod portion and wherein said coupling comprises one or more “click-fit” couplings for engagement with said rod.

In some arrangements said boss portion may be on one or other of said plate portions.

Preferably, the arrangement includes a locking means for locking one or other plate in a given angular position and said locking means comprises a protrusion on one plate portion for frictional engagement with a corresponding surface on said other plate portion. The locking means may include an expandable portion on one or other of said plate portions for expansion and frictional engagement with said other plate portion and said expandable portion may include a split portion having two or more segments and a biasing means for baising said segments apart and into engagement with said other plate portion.

Preferably, said upper or lower plate portions pivot about a common axis X.

In one arrangement the cage comprises a pair of spaced apart free standing side portions.

The side portions may be linked by a bridging portion at a lower edge thereof.

In one arrangement the implant includes a pair of axially spaced apart upper and lower plate portions having displaced axes of rotation.

Conveniently the implant includes an intermediate plate portion between said upper and lower plate portions and being mounted for pivotal rotation about said lower axis and may also include a plate motion limiter.

The limiter mentioned above may comprise an internal portion on said upper plate and an internal portion on said lower plate each of which are pivotal about a common axis and being movable between engaged an disengaged positions upon plate movement.

Although the following discussion focuses on spinal implants or prostheses, it will be appreciated that many of the principles may equally be applied to other bone structures within the human or animal body.

The present invention will now be more particularly described by way of example with reference to the accompanying drawings in which:

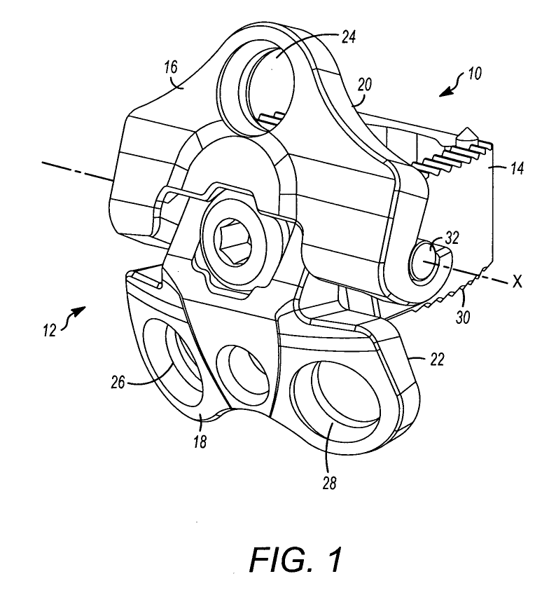

FIG. 1 is a general view of an assembled device according to a first aspect of the present invention;

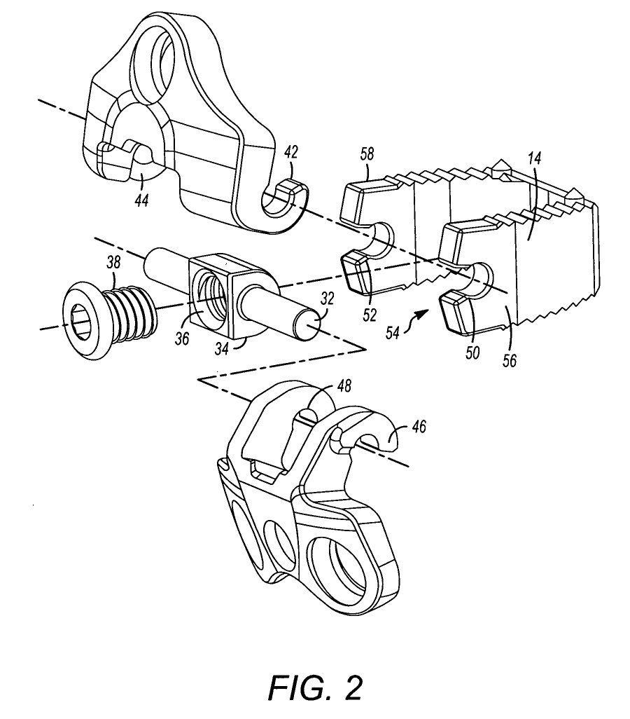

FIG. 2 is an exploded view of the device of FIG. 1;

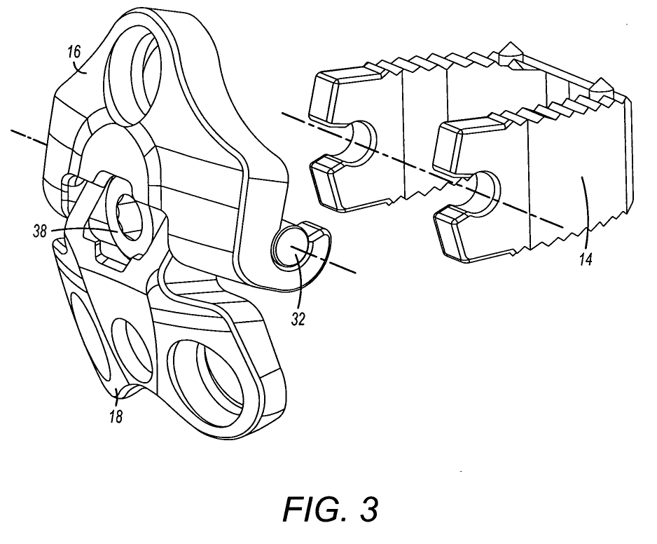

FIG. 3 is a partially assembled view of the device of FIG. 1;

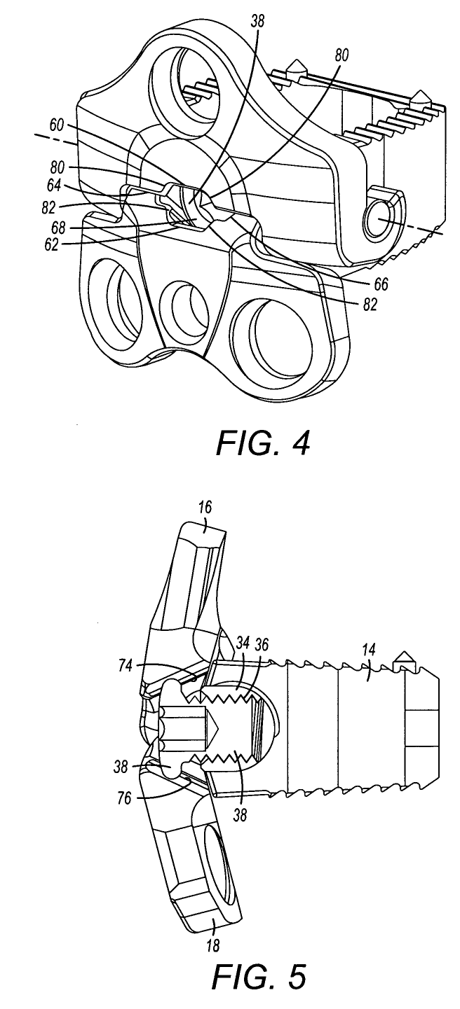

FIG. 4 is a general view of the device in the above drawings and illustrates the device in a first, straightened, attitude;

FIG. 5 is a cross-sectional view of the device of FIGS. 1 to 4 and illustrates the plates thereof angled relative to the cage;

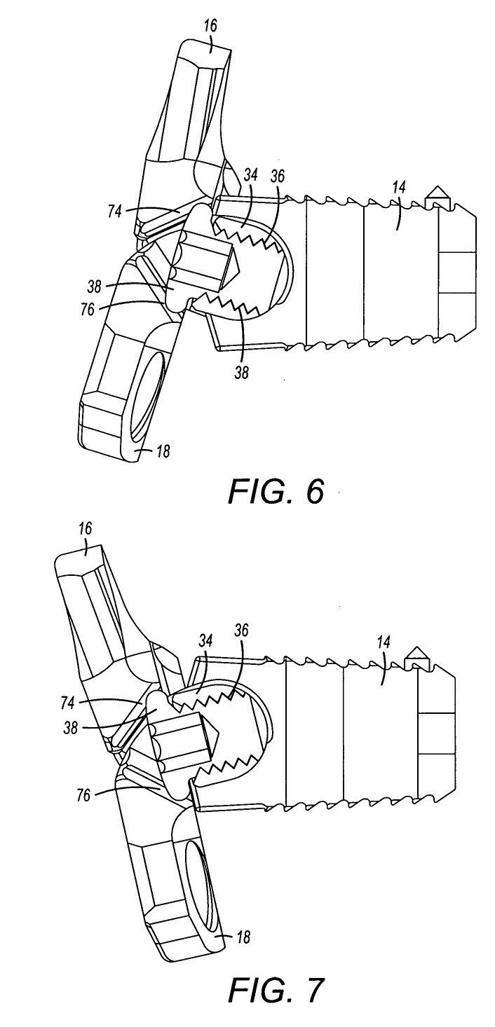

FIGS. 6 and 7 illustrate the degrees of freedom of movement possible with the device of the present invention;

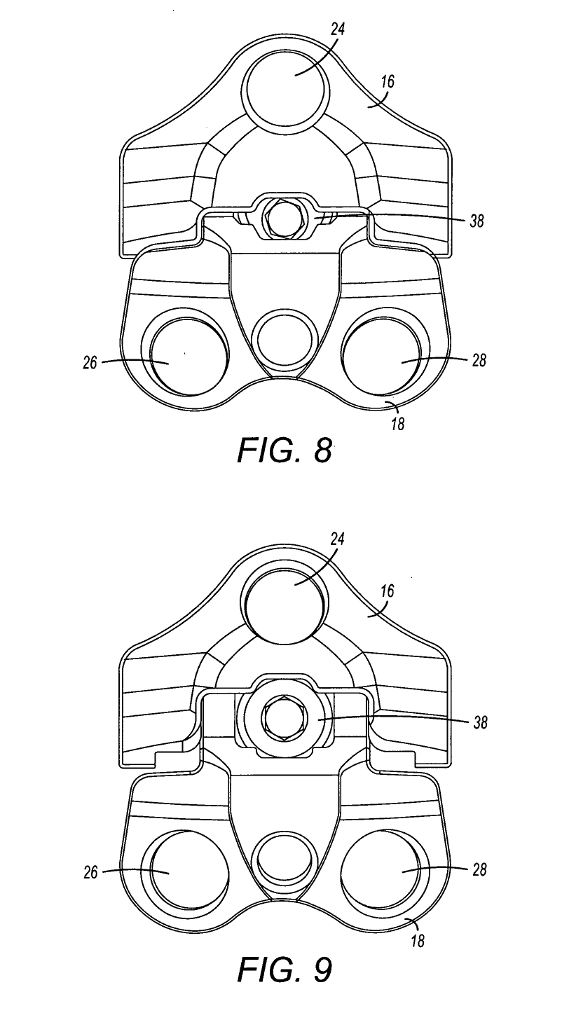

FIGS. 8 and 9 are elevations of the device and illustrate in more detail access to one portion thereof;

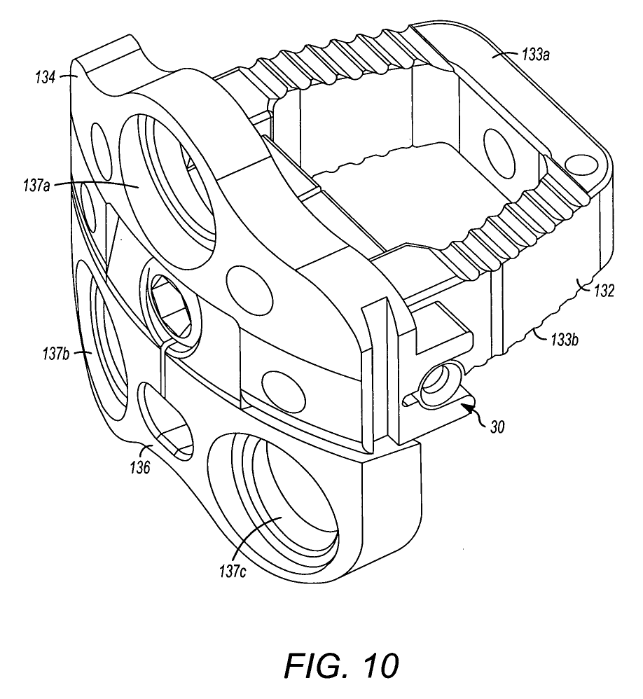

FIG. 10 is a general view of a second aspect of the present invention;

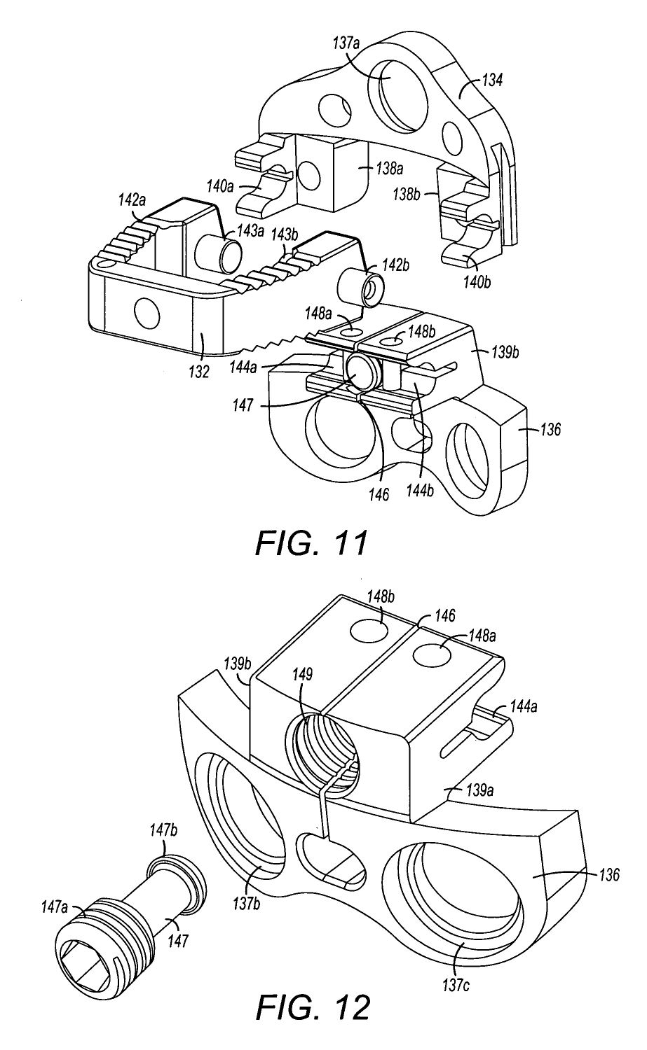

FIG. 11 is an exploded view of the device shown in FIG. 10;

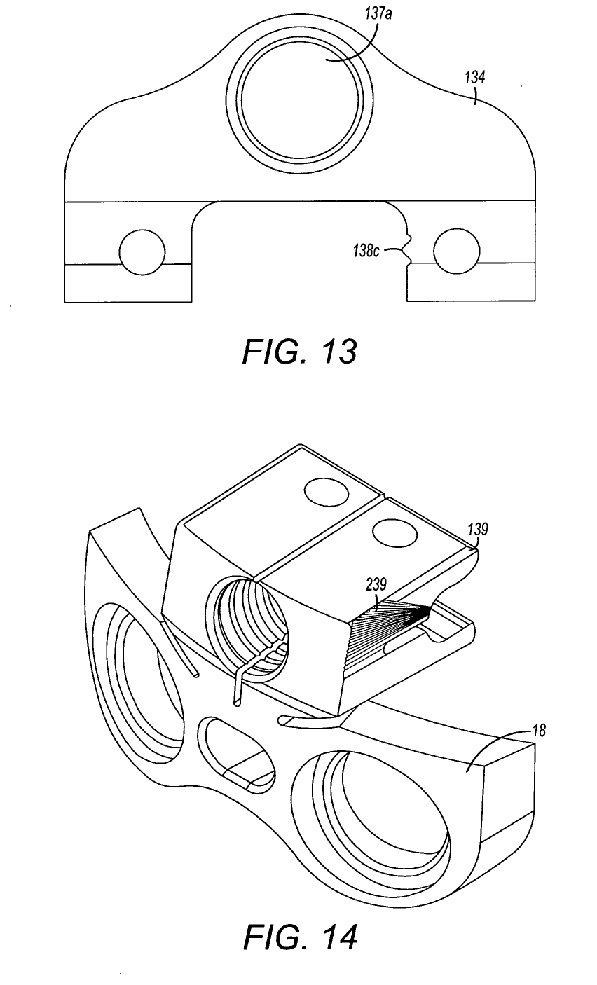

FIG. 12 is an enlarged view of a lower portion of the device shown in FIGS. 10 and 11;

FIG. 13 is a view of the upper portion;

FIG. 14 is a further view of the lower portion;

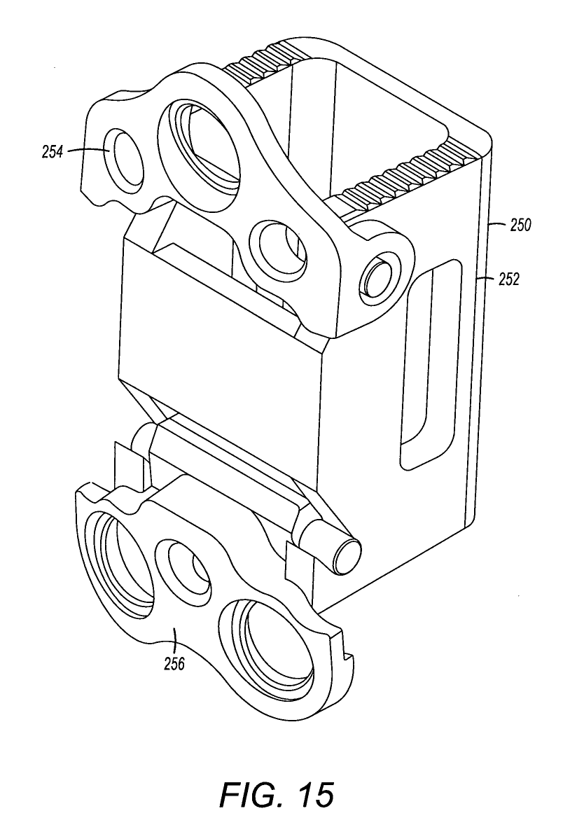

FIG. 15 is a general view of a still further embodiment of the present invention.

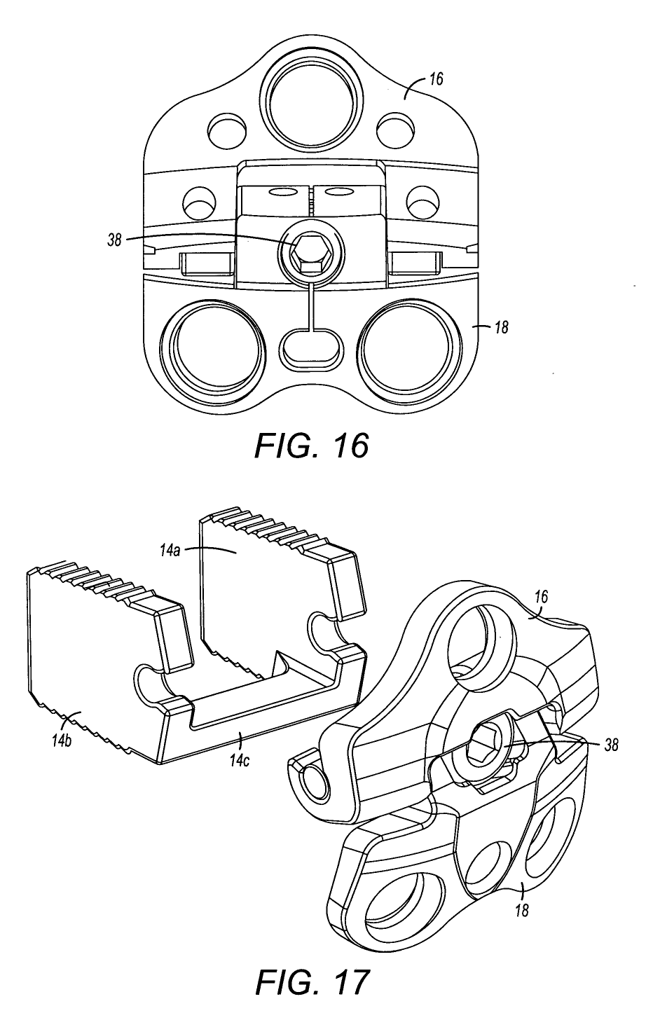

FIG. 16 is an elevation of a modified arrangement of the present invention;

FIG. 17 is an exploded view of the device of FIG. 16;

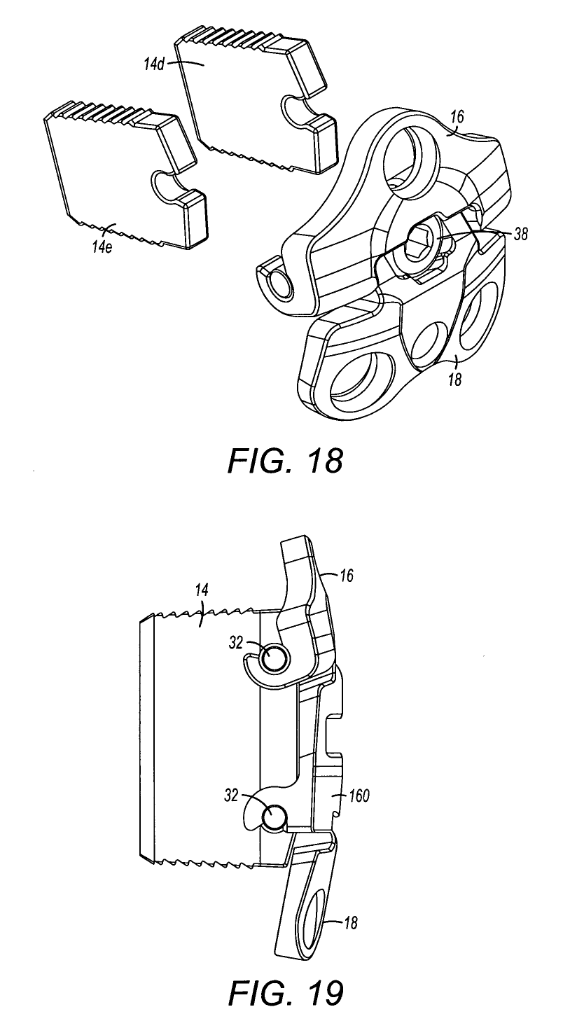

FIG. 18 is an exploded view of an alternative to the arrangement of FIGS. 16 and 17;

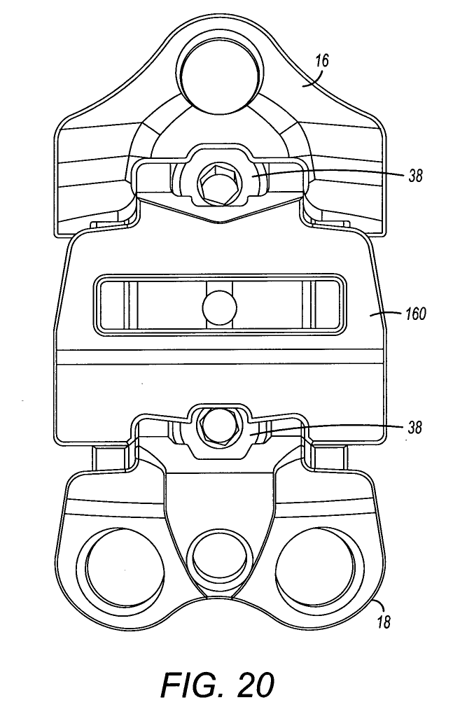

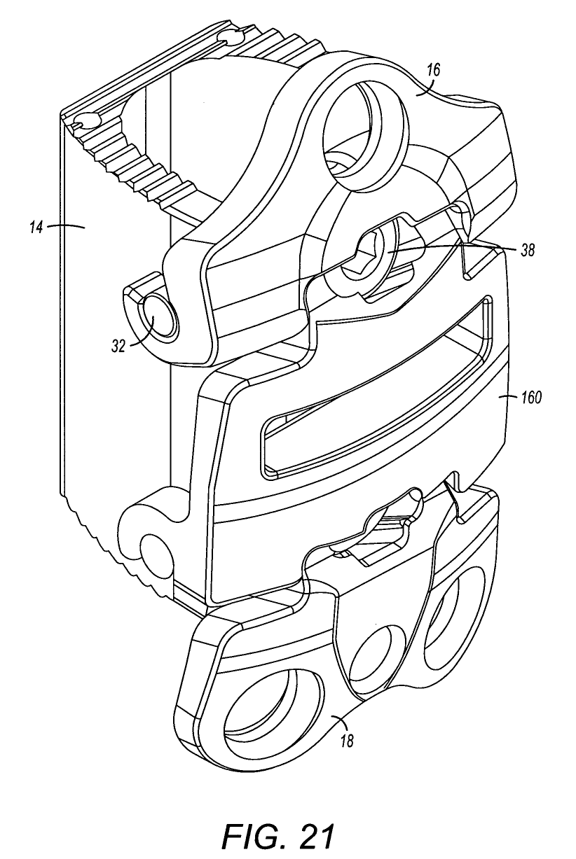

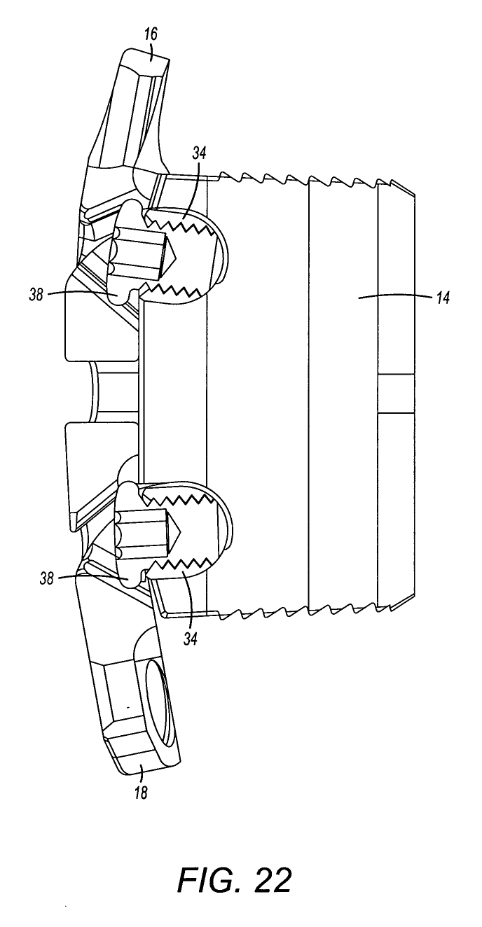

FIGS. 19 to 22 illustrate a still further modification to the present invention;

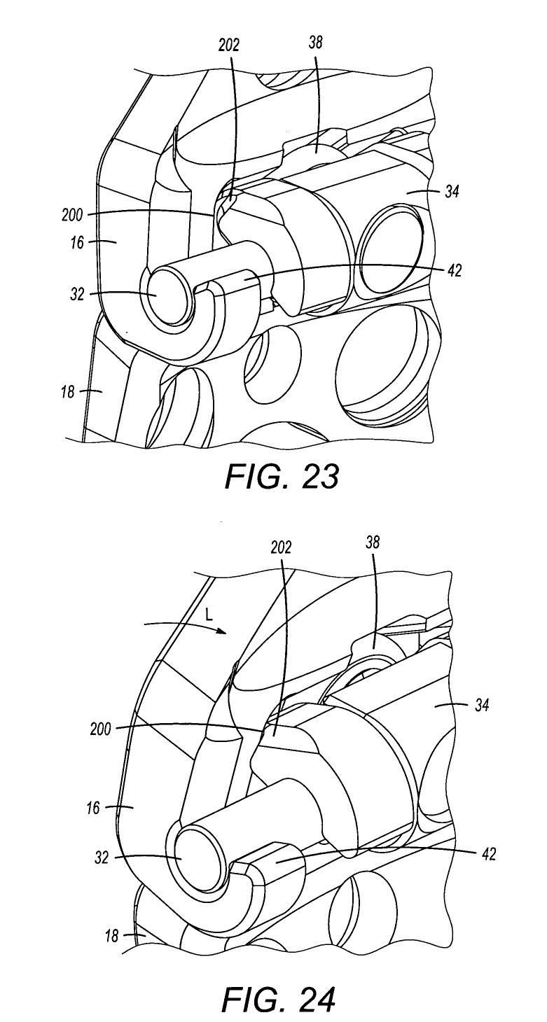

FIGS. 23 and 24 illustrate in more detail the plate stop mentioned above; and

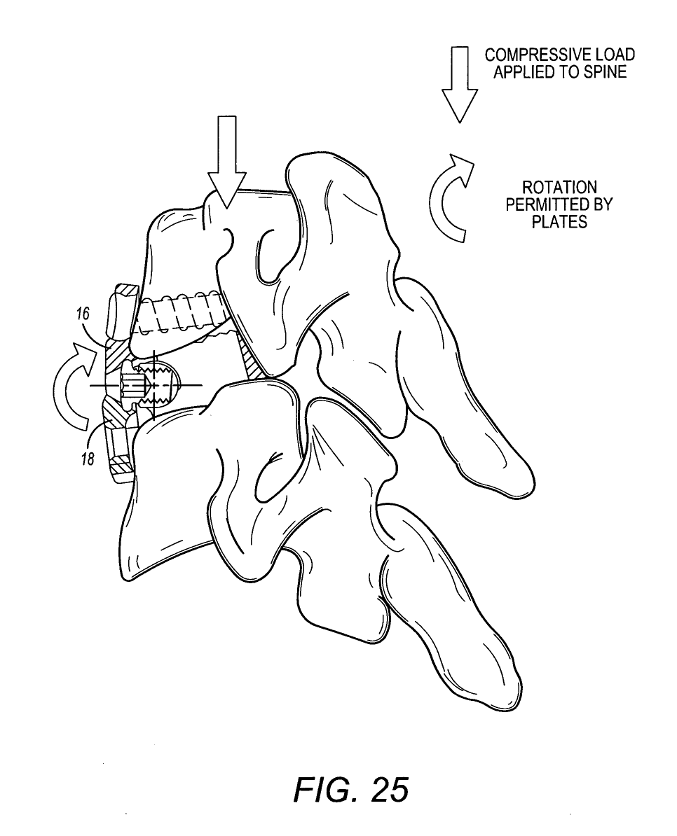

FIGS. 25 to 27 illustrate the locking mechanism in use.

Referring to FIG. 1, a spinal implant or prosthesis 10 includes a plate portion 12 for securing to the vertebrae and a cage 14 for insertion therebetween. The plate includes upper and lower portions 16, 18 having surfaces 20, 22 profiled to correspond with the profile of any bone material to which they are to be secured and one or more holes 24 26, 28 through which anchoring fasteners (not shown) may be inserted so as to secure the device to respective superior and inferior vertebral bodies (not shown). The fasteners may be screws, pins, staples, bollards or any other suitable fastening device. The upper and/or lower plate portions 16, 18 are pivotally coupled to the cage by means of a snap fit coupling shown generally at 30 and described in detail later herein. The coupling employs a pin arrangement 32 with which the cage portions 16, 18 engages and about which the portions may rotate or pivot (about axis X) so as to accommodate inter vertebral motion.

FIG. 2 illustrates the arrangement of FIG. 1 in an exploded manner from which it can be appreciated that the pin arrangement 32 extends laterally through the device and includes a mounting portion 34 having a threaded hole 36 for receiving a resistance bolt 38, the function of which will be described later. Also clear from FIG. 2 are semi-circular collar portions 42, 44, 46 and 48 on each of the upper and lower cage portions 16, 18 which provide a “click-fit” onto the pin arrangement 32. The manner of engagement will be well known to those skilled in the art but for the purpose of clarity, the arc of each collar extends slightly beyond 180 degrees such as to cause a slight interference to the insertion of the rod but insufficient resistance to prevent its insertion. As shown, the collars on the lower portion are place within the collars of the upper portion, although the opposite is also possible. Clearly shown in FIG. 2 is a pair of similar “snap-fit” couplings on the cage portion 14 and identified by references 50, 52. These couplings act in the manner as described above save for the fact that they couple the cage to the rod such as to allow all elements of the device to pivot about a common axis X. As shown, the cage comprises an open cage having an open anterior portion 54 and the couplings 50, 52 are provided at ends 56, 58 thereof. other arrangements may also be possible.

Referring now briefly to FIG. 4, it will be appreciated that the plate portions may be provided with a cut-out 60, 62 on confronting edges 64, 66 through which a surgeon may have access to the head portion 68 of any bolt 38 inserted into the boss portion, as will be described in more detail later herein.

FIGS. 5 to 7 are cross-sectional views of the device 10 and illustrate various positions of the plates relative to each other and the cage 14. From FIG. 5 it will be appreciated that the bolt head 68 may engage with portions of the inside surfaces 70, 72 of the plates. In more detail, one or each of the plates may be provided with an engagement surface 74, 76 against which the head of the bolt may engage when backed out of the boss portion 34. In operation, the bolt may be backed out of the boss 34 such that the head engages with one or other of the engagement portions and the degree of interference will dictate the degree of resistance the engagement will provide to plate movement. In effect, the arrangement forms a restraint to pivotal movement which varies from slight resistance to motion to full locking in a given angular position. In a preferred arrangement one or more of the plates are provided with a slot cut into the surface thereof and extending perpendicular to axis X which are shaped and positioned to engage with the head 68 at any angular position of said plates. The slot itself includes edges 80, 80a, 82, 82a (best seen in FIG. 4) which define cut-outs 83, 83a and which may provide the primary contact surface with the head of the bolt. FIG. 5 illustrates the plates and cage in an approximately perpendicular relationship with a slight curvature or angle between the plates themselves, such as one would need when restoring the natural curvature of a spine. FIGS. 6 and 7 illustrate two additional angular relationships where the plates having been locked in position are still capable of pivoting together relative to the cage 14 by virtue of the fact that the cage itself may pivot about axis X, with both plate elements moving as one with the boss portion 34. The total movement of the plates is limited by stops which are best seen with reference to FIGS. 25 to 27 below.

FIGS. 8 and 9 illustrate the plate portions in respective closed and open positions and from which it will be appreciated that the cut-outs 83, 83a provide access to the hex head fitting 84 which may be used to adjust the position of the bolt 38 as and when desired.

The cage portion 14 may be formed of a radio-translucent material, such as polyether-etherketone (PEEK), which means that the cage will not obscure inspection of the degree of bone growth inside the cage when imaged by x-rays. Additionally, the cage portion 14 may be formed of a bio-resorbable material. The bio-resorbable material is preferably osteo-conductive or osteo-inductive (or both). The plate portions 16, 18 may be formed of a metal such as titanium, or a super elastic or super plastic material, or may be a composite material, for example a long fibre composite material.

In use, the practitioner (for example an orthopaedic surgeon) after removal of a damaged disc inserts the cage portion 14 into the disc space between the adjacent superior and inferior vertebral bodies. Either before or after insertion the upper and lower plates 16, 18 are coupled to the cage 14 by pushing the U-shaped “click-fit” couplings onto the pin 32. The practitioner can then adjust the angles of the plates 16, 18 by pivotal movement to find the best anatomical position to secure the implant to the natural anatomy of the vertebral body. The bolt 38 is then adjusted such as to allow the head portion to engage with the inner surface of the plates and pivotal movement is thereby restrained.

Some practitioners prefer to allow some degree of movement between the implant and the adjacent vertebral body after implantation. In that case the bolt would not engaged with the back of the plates. Others prefer a more rigid implant, which does not allow relative movement between the vertebrae. This implant allows either preference.

FIGS. 10 to 13 illustrate a second embodiment of the present invention, a spinal implant 130 includes a cage portion 132, which is of a size and shape that corresponds to a space between two vertebral bodies (i.e. a disc space after removal of a disc). The cage 132 includes upper and lower surfaces 133a, 133b that are profiled to grip the bone surfaces of adjacent vertebral bodies (not shown). In the embodiment shown these are serrated surfaces to resist expulsion. Alternatively, these surfaces may have pyramid or other suitable shapes to resist expulsion. In addition, the implant includes an upper plate 134 and a lower plate 136. The upper and lower plates 134, 136 each contain one or more holes 137a, 137b and 137c through which anchoring fasteners (not shown) can be inserted so as to secure the implant to respective superior and inferior vertebral bodies (not shown). The fasteners may be screws, pins, staples, bollards or any other suitable fastening devices.

The upper and lower plates 134, 136 are coupled to the cage 132 by way of a snap or push-fit engagement, as described above. As can be seen in FIG. 11, the upper plate 134, is provided with a pair of spaced apart jaws 140a, 140b, which engage outwardly extending pins 142a, 142b on the cage 132. Similarly, the lower plate 136 has a pair of closely spaced jaws 144a, 144b, which engage inwardly extending pins 143a, 143b on the cage 132. In the assembled condition shown in FIG. 10, the plates 134, 136 are free to pivot to a certain degree on their respective pins 142, 143. In this embodiment, the two sets of pins 142, 143 are co-axial so as to provide a single common pivot axis for both plates. Each plate 134, 136 is free to pivot in a forward direction away from the position shown in FIG. 10.

The upper plate 134 has opposed inwardly-facing surfaces 138a, 138b which, in the assembled condition lie adjacent to corresponding outwardly-facing surfaces 139a, 139b on the closely-spaced jaws 144a and 144b of the lower plate 136. The closely spaced jaws 144a, 144b are separated by a narrow gap 146. A threaded hole 149, which is intersected by the narrow gap 146, extends into the lower plate 136 from the anterior side of the implant.

In one embodiment the cage portion 132 is formed of a radio-translucent material, such as polyether-etherketone (PEEK), which means that the cage will not obscure inspection of the degree of bone growth inside the cage when imaged by x-rays.

The cage portion 132 may be formed of a bio-resorbable material. The bio-resorbable material is preferably osteo-conductive or osteo-inductive (or both). The plate portions 134, 136 may be formed of a metal such as titanium, or a super elastic or super plastic material, or may be a composite material, for example a long fibre composite material.

In use, the practitioner (for example an orthopaedic surgeon) after removal of a damaged disc inserts the cage portion 132 into the disc space between the adjacent superior and inferior vertebral bodies. Either before or after insertion the upper and lower plates 134, 136 are coupled to the cage 132 by pushing the jaws 140a, 140b; 142a, 142b onto the respective pins, 142; 143. The practitioner can then adjust the angles of the plates 134, 136 by pivotal movement to find the best anatomical position to secure the implant to the natural anatomy of the vertebral body. A bolt 147, which may be a tapered bolt, or one that has a slightly larger thread diameter than the thread in the hole 149, is then advanced into the hole 149. As the bolt is tightened, the closely spaced jaws 144a, 144b are urged apart, so that the outwardly-facing surfaces 139 are urged into contact with the inwardly-facing surfaces 138 and pivotal movement is thereby restrained. The contact faces 138, 139 may have a roughened surface finish (lightly roughened or more heavily roughened). As discussed below in connection with FIG. 13, the contact faces may have grooves or splines machined into them which engage with each other to restrict movement once the locking screw is tightened. The implant is then secured to the vertebral body by means of fasteners (e.g. screws) positioned through the holes 137a, 137b. The fasteners preferably incorporate a locking device that prevents the screws backing out of the implant (as shown, for example, in FIG. 12 and described below).

Some practitioners prefer to allow some degree of movement between the implant and the adjacent vertebral body after implantation. In that case the both inserted into the hole 149 would not be fully tightened. Others prefer a more rigid implant, which is firmly locked to the adjacent vertebral body. The implant 130 allows either preference.

FIG. 12 shows the lower plate portion 136 in more detail, together with a securing bolt 147. As a threaded portion 147a, the securing bolt has a forwardly extending bollard-shaped portion 147b. Each of the jaws 144a, 144b has a downwardly extending hole 148a, 148b at a location forward of the threaded portion of the hole 149. After inserting the securing bolt 147 and tightening this to the required degree, pins can be inserted into the downwardly extending holes 148a, 148b to lock the securing bolt in place. The pins engage behind the head of the bollard-shaped portion 147b to prevent the securing bolt 147 backing out of the hole 149.

FIG. 13 shows an alternative plate having on one of its inwardly-facing contact surfaces 138 a ridge 138c. The plate of FIG. 14 has on one of its outwardly-facing contact surfaces 139 having a series of splines 239. When assembled the splines 239 and ridge 138a contact each other, the effect is to provide greater friction between the plates than would occur with smooth contact surfaces 138, 139. Thus, as the bolt in hole 149 tightened, pivotal movement is more greatly restrained.

The implant described above in connection with FIGS. 10 to 14, provide a unitary device that is inserted in one plane and is self centering, is conformable to surrounding anatomy, matches anatomical geometry, and matches natural anterior anatomical load constraints.

FIG. 15 depicts another spinal implant 250, similar in concept to the implant described above. The implant 250 is configured for replacement of part of or one or more entire vertebral bodies and discs, and includes an elongate cage portion 252 coupled to an upper plate 254 and a lower plate 256. In this case the plates 254, 256 are coupled to the cage to allow pivotal movement about separate pivot axes close to each end of the cage 252. This pivot action prevents the need for the surgeon to intra-operatively form the plates to conform to the anatomy of the vertebral body.

In the embodiments of FIGS. 10 to 14, the tops of the upper plates 134, 254 are shaped to correspond to the bottoms of the lower plates 136, 256, in order to allow a series of the implants to be closely stacked along the spine without interfering with each other. It will be appreciated that, instead of having one superior hole and two inferior holes in the implant as shown in the drawings, the implant may have two superior and one inferior holes, or may be adapted to have two superior holes and two inferior holes. Additionally, an arrangement with just a single hole in each upper and lower plate may be adopted.

FIG. 16 illustrates a still further view of the present invention whilst FIG. 17 illustrates an alternative form of cage 14 in which the cage comprises two distinct side portions 14a, 14b linked by a minor cross-member 14c. Such an arrangement would allow for relatively easy installation of a large amount of bone growth material and allows for better access during insertion of the cage and during any subsequent revision that may be necessary.

FIG. 18 illustrates a still further arrangement in which the cage is actually split into two distinct portions 14d and 14e, each of which is provided with a “click-fit” coupling as described above. Such an arrangement provides even better access and ease of installation as each side can be positioned independent of the other whilst they are stabilised and held rigid when the plates are coupled to the cages via the “click-fit” fitting.

FIGS. 19 to 22 provide a vertebreal replacement arrangement comprising a tall cage sized suitably for the replacement of a vertebra and having upper and lower plates 16, and 18 as described above and each being provided with a boss portion, bolt 38 and engagement surfaces 74, 76 on the plate portion associated therewith. The mounting on pin 32 is the same as above in that it employs a “click-fit” coupling of the cage plate to the pin associated with the cage and the control of angular plate position or freedom of movement is controlled by the interaction of the bolt 38 and the plates themselves, as described above. It will, however be noted that the arrangement of these figures provides a pair of pins identified as upper pin 32a and lower pin 32b and that the upper and lower plates are mounted on their own pin. The lower plate portion 18 may comprise two plates 18a, 18b with the upper plate 18b effectively forming an intermediate plate 160 and each of these plates may be mounted on the lower pin 32b as described above with reference to FIGS. 1 to 9. When an intermediate plate is provided it may also be profiled at an upper edge 160a so as to accommodate or reflect the profile of the lower edge 16b of the upper plate 16 and to allow access to the bolt 38.

Installation and locking of the arrangement described immediately above is similar that described with reference to the embodiments of FIGS. 1 to 18 save for the fact that the plates will be assembled to the boss portion and then click-fitted to the cage and this will probably have to be done before the implant is inserted into the patient.

Reference is now made to FIGS. 23 and 24 which illustrate a stop arrangement which prevents excessive plate movement. From these figures it will be appreciated that an inner surface portion 200 on the upper plate portion 16 is located adjacent to an outer surface portion 202 on a lower plate portion 18 and by virtue of the fact that the plates each pivot about pin 32 the surfaces will come into contact with each other as and when the upper plate moves excessively in the direction of arrow L. This arrangement will prevent excessive bending or rotation of the pates 16, 18 relative to each other. This prevents the front opening formed by the cut-outs 80, 82 from opening wide enough to permit the head of the bolt 38 from passing through. This acts as an anti-back-out measure to prevent the bolt escaping from the assembly and into the patient's body

Once implanted, with the plates securely fastened to the vertebral bodies and the central bolt engaged with the posterior aspect of the plates, the present invention provides beneficial functions as described below with reference to FIGS. 25 to 27.

In the neutral position, i.e. with the patient's head in an upright orientation, the present invention provides support to the spinal column. In addition, because the central bolt does not block inward rotation of the plates, the small amount of movement permitted by the design allows compressive loading to be applied directly to the cage and any bone graft or bone substitute material contained within. This compressive loading encourages osteoblast activity and hence new bone formation, in accordance with Wolff's Law. See FIG. 25.

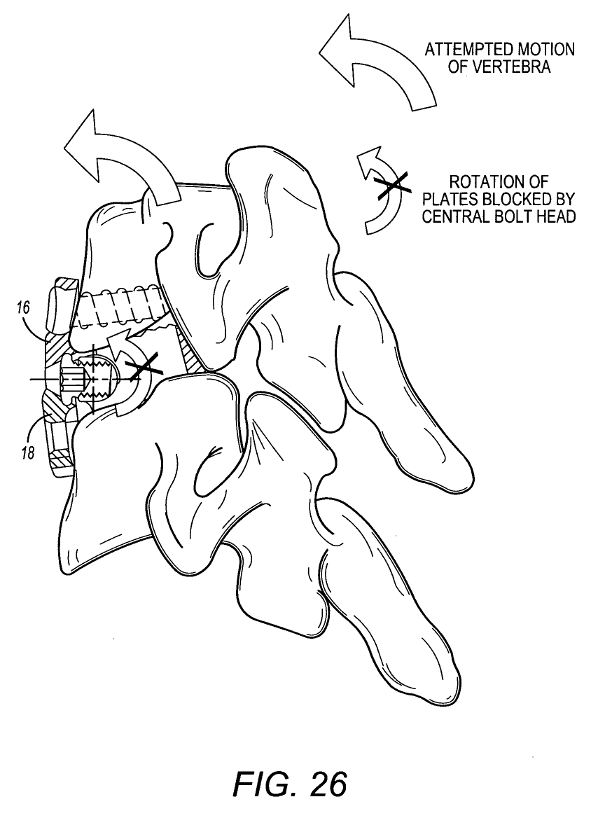

In flexion, i.e. as the patient brings their chin towards their chest, the natural motion of the vertebrae would be to pivot about a centre of rotation located towards the anterior aspect of the intervertebral joint, reducing the lordotic angle and allowing the neck to curve forwards. This is potentially the most damaging movement for an intervertebral joint in the early stages of fusion, as the bone graft or bone substitute material is subjected to a tensile load. Bone, generally, is strong in compression but considerably weaker in tension, and is especially vulnerable during the process of incorporation of graft material into a stable arthrodesis. The present invention protects against possible damage to the graft site because the only axis of rotation available to the vertebral bodies is about the axis of the pivot pin. However, to allow this type of motion the plates would have to rotate outwards, and movement in this case is blocked by the head of the central bolt, which prevents the gap between the plates from closing. See FIG. 26.

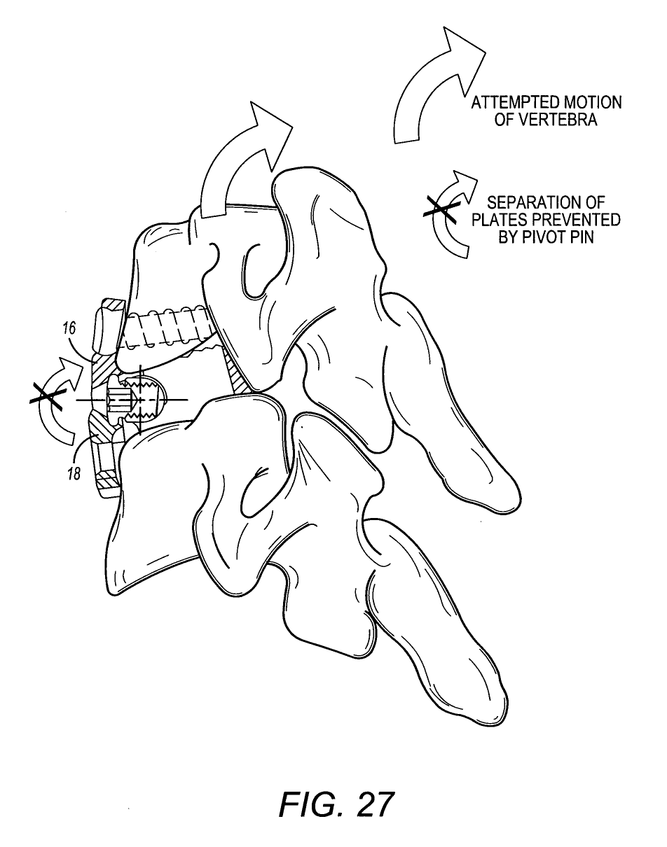

In extension, i.e. as the patient tilts their head backwards, the natural motion of the vertebrae would be to pivot about a centre of rotation located towards the posterior aspect of the intervertebral joint, increasing the lordotic angle and allowing the neck to curve backwards. In this case, the present invention protects against movement because the vertebrae are likely attempt to pivot about the posterior edges of the intervertebral cage. This movement would require the anterior elements of the fused joint to move apart (i.e. the superior plate moves away from the inferior plate in an upwards direction). Due to the fact that both plates hook around the pivot pin, the assembled device of the present invention acts as a tension band in this application, and prevents separation of the plates and, therefore, separation of the vertebral bodies from the bone graft material. See FIG. 27.

Those skilled in the art will appreciate that the “click-fit” arrangement of the present invention may allow a surgeon to install the cage first and then simply “click-fit” the outer plate portions before adjusting the degree of allowed motion of one or other or both thereof by adjusting bolt 38. Alternatively, the bolt can be adjusted after the plate portions have been adjusted individually to a desired profile. Still further, the bolt arrangement allows for the provision of a different degree of motion restriction for each plate portions or the provision of the same degree of restriction. The open ended nature of the cage allows for the easy insertion of bone growth stimulation material and may assist with the establishment of a strong vertebra to vertebra fusion. Still further, the separate side cage portion as shown in FIG. 18 are relatively easily inserted and may assist a surgeon establish and position a cage when access is limited or difficult. The fact that the cage and the plate pivot about a common axis (X) will assist with the generation of an improved load transfer.

In addition to the above, it has been observed that the arrangement of the present invention has a relatively low profile external to that of the vertebrae to which it is to be fitted and this may enhance the longevity of the device within a patient.

Still further, it will be appreciated that the simple nature of the angular adjustment and the fact that it can be done “in situ” will eliminate the metal bending step common to many other techniques and may well provide a much better degree of conformality to the vertebral structure

1. A spinal implant comprising upper and lower plate portions for securing to vertebral bodies and a cage portion for securing between vertebral bodies, wherein each of said plate portions and said cage portion are independently pivotal about a common axis X such as to allow for flexibility between vertebrae.

2. A spinal implant as claimed in claim 1 and including a resistance means for resisting pivotal movement of said one or more upper or lower portions.

3. A spinal implant as claimed in claim 1, wherein one or more of said plate portions includes an engagement surface with which said resistance means engages.

4. A spinal implant as claimed in claim 2, wherein said implant includes a mounting portion and wherein said resistance means comprises a bolt having an engagement portion for frictional engagement with said one or more plate portions and a bolt thread for engagement with said mounting portion.

5. A spinal implant as claimed in claim 4, wherein said resistance means comprises a bolt head portion of said bolt and said one or more plates include a channel having edges therein which form an engagement surface and which, in operation, engage with said bolt head.

6. A spinal implant as claimed in claim 1, wherein one or more of said plate portions include a semi-circular collar portion, said axis comprises a rod portion and said semi-circular collar portion engages around said rod portion for pivotal movement relative thereto.

7. A spinal implant as claimed in claim 6 wherein said upper and lower plate portions each include semi-circular collar portions and each engage with said rod portion for pivotal movement relative thereto.

8. A spinal implant as claimed in claim 1 and including a mounting boss for receiving the bolt thread of said bolt.

9. A spinal implant as claimed in claim 8 wherein said boss includes a rod portion onto which one or more of said plate portions are mounted for pivotal movement relative thereto.

10. A spinal implant as claimed in claim 1 in which the one or more engagement surfaces on the one or more plate portions are on an inner surface of said plate or plates.

11. A spinal implant as claimed in claim 6, wherein one or more of said upper or lower plates include a “click-fit” fitting for engagement with said rod.

12. A spinal implant as claimed in claim 1, wherein said upper and lower plates include a cut-out portion on confronting edges thereof, said bolt head includes an axis which extends through said cut-out portion and includes an engagement feature accessible through said cut-out portion.

13. A spinal implant as claimed in claim 12 wherein said bolt head has a diameter greater than the size of said cut-out.

14. A spinal implant as claimed in claim 7, wherein said upper plate and said lower plate each comprise a pair of collar portions and wherein one pair are spaced apart more than the other pair such as to allow one pair to nestle between the other pair when engaged with said rod.

15. (canceled)

16. (canceled)

17. (canceled)

18. (canceled)

19. A spinal implant as claimed in claim 1, wherein said cage portion comprises an open ended cage having side arms and wherein said side arms each include a coupling for coupling said cage to said plates.

20. A spinal implant as claimed in claim 19, wherein said implant includes a rod portion about which said plates pivot and said coupling comprises one or more cut-outs for engagement with the rod portion.

21. A spinal implant as claimed in claim 20 wherein said coupling comprises one or more “click-fit” couplings for engagement with said rod.

22. A spinal implant as claimed in claim 9, wherein said boss portion is on one or other of said plate portions.

23. A spinal implant as claimed in claimed in claim 1 and including locking means for locking one or other plate in a given angular position.

24. A spinal implant as claimed in claim 23 wherein said locking means comprises a protrusion on one plate portion for frictional engagement with a corresponding surface on said other plate portion.

25. A spinal implant as claimed in claim 23, wherein said locking means includes an expandable portion on one or other of said plate portions for expansion and frictional engagement with said other plate portion.

26. A spinal implant as claimed in claim 25 wherein said expandable portion includes a split portion having two or more segments and a biasing means for baising said segments apart and into engagement with said other plate portion.

27. (canceled)

28. A spinal implant as claimed in claim 1 in which the cage comprises a pair of spaced apart free standing side portions.

29. A spinal implant as claimed in claim 28 wherein said side portions are linked by a bridging portion at a lower edge thereof.

30. A spinal implant as claimed in claim 1 and including a pair of axially spaced apart upper and lower plate portions having displaced axes of rotation.

31. A spinal implant as claimed in claim 30 and including an intermediate plate portion between said upper and lower plate portions and being mounted for pivotal rotation about said lower axis.

32. A spinal implant as claimed in claim 1, and including a plate motion limiter.

33. A spinal implant as claimed in claim 32 wherein said limiter comprises an internal portion on said upper plate and an internal portion on said lower plate each of which are pivotal about a common axis and being movable between engaged an disengaged positions upon plate movement
